(19) United States
(12) Patent Application Publication
(10) Pub. No.: US 2019/0184123 A1
(43) Pub. Date: Jun. 20, 2019

(54) GROUNDING POSITIVE AIRWAY PRESSURE MASKS AND HEADGEAR

(71) Applicant: KONINKLIJKE PHILIPS N.V., Eindhoven (NL)

(72) Inventors: LAUREN PATRICIA CHODKOWSKI, PITTSBURGH, PA (US); MICHAEL THOMAS KANE, HARRISON CITY, PA (US)

(21) Appl. No.: 16/224,848

(22) Filed: Dec. 19, 2018

Related U.S. Application Data
(60) Provisional application No. 62/608,077, filed on Dec. 20, 2017.

(51) Int. Cl. A61M 16/06; A61M 16/08

(52) U.S. Cl. CPC A61M 16/0666; A61M 16/0616; A61M 2016/0018; A61M 16/0633; A61M 16/0875

(57) ABSTRACT

An interface device for delivering a flow of a treatment gas to an airway of a patient includes: a conductive element positioned on a surface such that the conductive element directly contacts the patient when the interface device is positioned on the patient for delivering the flow of the treatment gas to the airway of the patient; and a conductive pathway extending from a first end which is electrically connected to the conductive element to an opposite second end which is structured to be electrically connected to earth ground.

### CROSS-REFERENCE TO RELATED APPLICATIONS

This patent application claims the priority benefit under 35 U.S.C. § 119(e) of U.S. Provisional Application No. 62/608,077, filed on Dec. 20, 2017, the contents of which are herein incorporated by reference.

### BACKGROUND OF THE INVENTION

### 1. Field of the Invention

The present invention pertains to system for delivering a flow of treatment gas to the airway of a patient and more particularly to patient interface devices for use in such systems that include a grounding element for contacting and grounding the patient.

### 2. Description of the Related Art

Many individuals suffer from disordered breathing during sleep. Sleep apnea is a common example of such sleep disordered breathing suffered by millions of people throughout the world. One type of sleep apnea is obstructive sleep apnea (OSA), which is a condition in which sleep is repeatedly interrupted by an inability to breathe due to an obstruction of the airway; typically the upper airway or pharyngeal area. Obstruction of the airway is generally believed to be due, at least in part, to a general relaxation of the muscles which stabilize the upper airway segment, thereby allowing the tissues to collapse the airway. Another type of sleep apnea syndrome is a central apnea, which is a cessation of respiration due to the absence of respiratory signals from the brain's respiratory center. An apnea condition, whether obstructive, central, or mixed, which is a combination of obstructive and central, is defined as the complete or near cessation of breathing, for example a 90% or greater reduction in peak respiratory air-flow.

Those afflicted with sleep apnea experience sleep fragmentation and complete or nearly complete cessation of ventilation intermittently during sleep with potentially severe degrees of oxyhemoglobin desaturation. These symptoms may be translated clinically into extreme daytime sleepiness, cardiac arrhythmias, pulmonary-artery hypertension, congestive heart failure and/or cognitive dysfunction. Other consequences of sleep apnea include right ventricular dysfunction, carbon dioxide retention during wakefulness, as well as during sleep, and continuous reduced arterial oxygen tension. Sleep apnea sufferers may be at risk for excessive mortality from these factors as well as by an elevated risk for accidents while driving and/or operating potentially dangerous equipment.

Even if a patient does not suffer from a complete or nearly complete obstruction of the airway, it is also known that adverse effects, such as arousals from sleep, can occur where there is only a partial obstruction of the airway. Partial obstruction of the airway typically results in shallow breathing referred to as a hypopnea. A hypopnea is typically defined as a 50% or greater reduction in the peak respiratory air-flow. Other types of sleep disordered breathing include, without limitation, upper airway resistance syndrome (UARS) and vibration of the airway, such as vibration of the pharyngeal wall, commonly referred to as snoring.

It is well known to treat sleep disordered breathing by applying a continuous positive air pressure (CPAP) to the patient's airway. This positive pressure effectively “splints” the airway, thereby maintaining an open passage to the lungs. It is also known to provide a positive pressure therapy in which the pressure of gas delivered to the patient varies with the patient's breathing cycle, or varies with the patient's breathing effort, to increase the comfort to the patient. This pressure support technique is referred to as bi-level pressure support, in which the inspiratory positive airway pressure (IPAP) delivered to the patient is higher than the expiratory positive airway pressure (EPAP). It is further known to provide a positive pressure therapy in which the pressure is automatically adjusted based on the detected conditions of the patient, such as whether the patient is experiencing an apnea and/or hypopnea. This pressure support technique is referred to as an auto-titration type of pressure support, because the pressure support device seeks to provide a pressure to the patient that is only as high as necessary to treat the disordered breathing.

Pressure support therapies as just described involve the placement of a patient interface device including a mask component having a soft, flexible sealing cushion on the face of the patient. The mask component may be, without limitation, a nasal mask that covers the patient's nose, a nasal/oral mask that covers the patient's nose and mouth, or a full face mask that covers the patient's face. Such patient interface devices may also employ other patient contacting components, such as forehead supports, cheek pads and chin pads. The patient interface device is typically secured to the patient's head by a headgear component. The patient interface device is connected to a gas delivery tube or conduit and interfaces the pressure support device with the airway of the patient, so that a flow of breathing gas can be delivered from the pressure/flow generating device to the airway of the patient.

Patients suffering from sleep apnea commonly suffer from one or more comorbidities. Accordingly, additional wellness applications linked with a patient's CPAP device may provide additional health benefits to the patient while also helping to increase compliance.

### SUMMARY OF THE INVENTION

As one aspect of the present invention, an interface device for delivering a flow of a treatment gas to an airway of a patient is provided. The interface device comprises: a conductive element positioned on a surface such that the conductive element directly contacts the patient when the interface device is positioned on the patient for delivering the flow of the treatment gas to the airway of the patient; and a conductive pathway extending from a first end which is electrically connected to the conductive element to an opposite second end which is structured to be electrically connected to earth ground.

The conductive element may comprise a conductive wrap selectively coupled to a portion of the interface device.

The interface device may further comprise a patient sealing assembly and the conductive element may comprise a cover disposed on the sealing assembly. The cover may be disposed only on a portion of the sealing assembly. The cover may be disposed about generally the entirety of the sealing assembly.

As another aspect of the present invention, a sealing assembly for use in delivering a flow of a treatment gas to an airway of a patient is provided. The sealing assembly comprises: a body portion structured to be coupled to a head of a user; a sealing portion having a surface structured to sealingly engage the face of a user at or about an airway of the user; and a conductive element positioned on the surface such that the conductive element directly contacts the patient when the interface device is positioned on the patient, wherein the conductive element is structured to be electrically coupled to ground.

The sealing portion may be formed from a conductive material and the conductive element may be a portion of the sealing portion. The conductive material may comprise a conductive silicone.

The body portion may be formed from a non-conductive material other than a lead formed therein which is formed from a conductive material which is electrically connected to the conductive element.

The conductive element may comprise a cover disposed on the sealing assembly. The cover may be disposed only on a portion of the sealing assembly. The cover may be disposed about generally the entirety of the sealing assembly.

As yet a further aspect of the present invention, an airway pressure support system for use in delivering a flow of breathing gas to the airway of a patient is provided. The pressure support system comprises: a pressure/flow generating device which is structured to generate the flow of breathing gas; a patient interface device structured to be disposed on the head of the patient, the patient interface device comprising: a headgear for securing the patient interface device to the head of the patient; and a patient sealing assembly which is structured to sealingly engage the face of the patient at or about an airway of the patient; a delivery conduit coupled between the pressure/flow generating device and the patient interface device, the delivery conduit structured to communicate the flow of breathing gas from pressure generating device to the patient interface device; a conductive element positioned on a surface of the patient interface device such that the conductive element directly contacts the patient when the interface device is positioned on the patient; and a conductive pathway which extends from the conductive element to a second end which is structured to be electrically connected to earth ground.

The conductive pathway may be a portion of a grounding system which also comprises a processing unit; the conductive pathway may include a first resistor, a switch, and a second resistor which are all electrically connected in series; and the switch may be selectively switched between an open position and a closed position by the processing unit.

The processing unit may be associated with the control/operation of the pressure/flow generating device.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a simplified diagram of an airway pressure support system according to an exemplary embodiment of the present invention;

FIG. 2 is a schematic representation of a grounding system according to an exemplary embodiment of the present invention;

FIG. 3 is a partially schematic, isometric view of a patient sealing assembly according to an exemplary embodiment of the present invention;

FIG. 4 is partially schematic, isometric view of an airway pressure support system according to an exemplary embodiment of the present invention including the patient sealing assembly of FIG. 3;

FIG. 5 is a partially schematic, isometric view of a patient interface device according to an exemplary embodiment of the present invention;

FIG. 6 is a partially schematic, isometric view of a cover according to an exemplary embodiment of the present invention for use on a patient sealing assembly; and

FIG. 7 is a partially schematic, isometric view of a patient interface device according to another exemplary embodiment of the present invention.

### DETAILED DESCRIPTION OF EXEMPLARY EMBODIMENTS

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As used herein, the statement that two or more parts or components “engage” one another shall mean that the parts exert a force against one another either directly or through one or more intermediate parts or components. As used herein, the term “number” shall mean one or an integer greater than one (i.e., a plurality).

As used herein, “conductive material” means a material which has a resistance of no more than about 2 ohms. Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

Ground is the concept of conductive contact of the human body with the surface of the earth. The idea is that the free electrons on the earth's surface can balance out electrochemical physiological processes that can lead to improved healing, reduction in inflammation, better sleep, and a reduction in overall pain. Most grounding or “earthing” devices are worn or used during sleep. Some of the most common applications are bracelets, conductive sheets or mattresses. Because compliant sleep apnea patients already sleep with a CPAP mask contacting their face, additional benefits can be obtained by adding conductive materials to masks and headgear to improve overall sleep and wellness.

Embodiments of the present invention are directed generally to patient interface devices and components thereof which provide an arrangement for grounding the user of the device while receiving treatment. Embodiments of the present invention include the means to measure, monitor and control the discharge of electrons through such grounding. This is accomplished by a computer or logic processing unit.

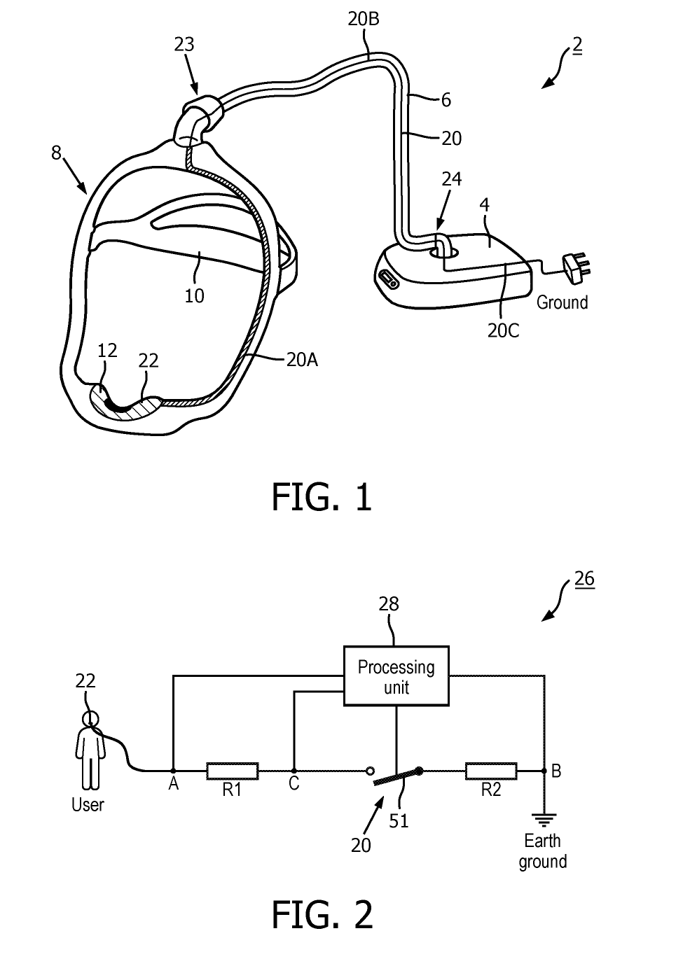

An example airway pressure support system 2 according to one particular, non-limiting exemplary embodiment of the present invention is shown in FIG. 1. System 2 includes a pressure/flow generating device 4, a delivery conduit 6, and a patient interface device 8 structured to be disposed on the head of a patient. Pressure generating device 4 is structured to generate a flow of breathing gas which may be heated and/or humidified. Pressure generating device 4 may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 6 is structured to communicate the flow of breathing gas from pressure generating device 4 to patient interface device 8. Delivery conduit 6 and patient interface device 8 are often collectively referred to as a patient circuit. Patient interface device 8 includes a headgear 10 for securing patient interface device 8 to the head of a patient (not shown).

A BiPAP® device is a bi-level device in which the pressure provided to the patient varies with the patient's respiratory cycle, so that a higher pressure is delivered during inspiration than during expiration. An auto-titration pressure support system is a system in which the pressure varies with the condition of the patient, such as whether the patient is snoring or experiencing an apnea or hypopnea. For present purposes, pressure/flow generating device 4 is also referred to as a gas flow generating device, because flow results when a pressure gradient is generated. The present invention contemplates that pressure/flow generating device 4 is any conventional system for delivering a flow of gas to an airway of a patient or for elevating a pressure of gas at an airway of the patient, including the pressure support systems summarized above and non-invasive ventilation systems. Although described herein in example embodiments wherein a pressurized flow of gas is utilized, it is to be appreciated that embodiments of the invention as described herein could also be readily employed in other generally non-pressurized applications (e.g., without limitation, in high flow therapy applications).

Patient interface device 8 includes a patient sealing assembly 12 which is structured to sealingly engage the face of a user at or about an airway of the user. In the illustrated exemplary embodiment, patient sealing assembly is a nasal cushion, however, it is to be appreciated that other types of patient sealing assemblies, such as, without limitation, a nasal/oral mask, a nasal pillows mask, or any other arrangements which facilitate the delivery of the flow of breathing gas to the airway of a patient may be substituted for patient sealing assembly 12 while remaining within the scope of the present invention. It is also to be appreciated that headgear 10 is provided solely for exemplary purposes and that any suitable headgear arrangement may be employed without varying from the scope of the present invention.

System 2 includes a conductive pathway, shown generally by numeral 20, which extends from a conductive element 22 included as a part of patient interface device 8, along delivery conduit 6, and through pressure generating device 4 to ground. As will be appreciated from the further discussion below, conductive pathway 20 may be formed from a number of suitable conductive elements which may be formed from any suitable conductive material or materials and electrically connected via any suitable coupling arrangement or arrangements. In the example shown in FIG. 1, conductive pathway 20 is generally formed from three segments, a first segment 20A, a second segment 20B, and a third segment 20C, which are electrically connected in series. More particularly, first segment 20A extends from conductive element 22 to a first junction 23 between patient interface device 8 and delivery conduit 6 where first segment 20A is electrically connected to a first end of second segment 20B; second segment 20B extends from first junction 23 to a second junction 24 between delivery conduit 6 and pressure/flow generating device 4 wherein a second end of second segment 20B is electrically connected to a first end of third segment 20C; third segment 20C extends from second junction 24 to Earth ground.

Conductive element 22 is positioned on, or as a portion of, a surface of patient interface device 8 such that conductive element 22 directly contacts the skin of a user when patient interface device 8 is positioned on the head of a user. In the example shown in FIG. 1, conductive element 22 is formed from a conductive silicone positioned as an outer surface of the nasal cushion. Conductive element 22 is electrically connected to first segment 20A which, in the example embodiment shown in FIG. 1, is also formed from a conductive silicone. As will be appreciated from the further discussion below, conductive element may be formed and or positioned in various ways without varying from the scope of the present invention.

FIG. 2 shows schematically a grounding system 26 according to one particular, non-limiting exemplary embodiment of the present invention which includes conductive pathway 20, such as described in conjunction with FIG. 1, and an associated processing unit 28 which is structured to monitor/control elements of conductive pathway 20. Processing unit 28 may be associated with the control/operation of pressure/flow generating device 4. Alternatively, processing unit 28 may be a dedicated processing device which is not related to the control/operation of pressure/flow generating device 4. In either case, processing unit 28 includes a processing portion which may be, for example, a microprocessor, a microcontroller or some other suitable processing device, and a memory portion that may be internal to the processing portion or operatively coupled to the processing portion and that provides a storage medium for data and software executable by the processing portion for controlling the operation of grounding system 26. The memory portion can be any of one or more of a variety of types of internal and/or external storage media such as, without limitation, RAM, ROM, EPROM(s), EEPROM(s), FLASH, and the like that provide a storage register, i.e., a machine readable medium, for data storage such as in the fashion of an internal storage area of a computer, and can be volatile memory or nonvolatile memory.

Continuing to refer to FIG. 2, conductive pathway 20 includes a first resistor R1, a switch S1, and a second resistor R2, which are all connected in series. Resistor R1 is a small (e.g., about 0.001 ohm) current sense resistor and second resistor R2 is a current limiting resistor (e.g., about 5 ohms). Alternatively, R2 may be replaced with a controllable variable resistor such as described below. When switch S1 is disposed in an open position (such as shown in FIG. 2), conductive pathway 20 between the user and ground is open, preventing electron flow from the patient. The voltage potential between points A and B can be monitored, such as by processing unit 26, as described below. When switch S1 is disposed in a closed position, conductive pathway 20 between the user and ground is closed.

In operation, when first connected, processing unit 28 begins by measuring the electrical potential difference between the user and earth ground (i.e., the voltage potential between points A and B). The potential voltage difference between the user and earth ground provides more information when combined with body weight of the user, which may be input to processing unit 28 via any suitable means. The next step involves the equalization of voltage potential between the user and earth ground. During this process, switch S1 is closed and the user's potential energy is brought into equilibrium with the Earth. This can be accomplished by wired connection or though some non-contact means. During the equalization process the number of electrons discharged by the user are monitored and the rate of equalization may be controlled. The number of electrons discharged by the user can be monitored by observing the voltage drop across the small first resistor R1 (i.e., voltage drop between points A and C). Monitoring the voltage drop across first resistor R1 allows one to monitor the current and thus the number of electrons passing through first resistor R1. As previously mentioned, second resistor R2 may be replaced by a controllable (e.g., by processing unit 28) variable resistor which may programmatically be used to provide therapeutic benefit to the user by limiting the rate at which electrons are exchanged. Another therapeutic step is to open switch S1 after a certain number of electrons have been exchanged in order to cease the flow of electrons.

Combining the initial potential difference along with the number of electrons exchanged during equalization provides a more complete picture of the number of free radicals prior to grounding of the patient. These values may be stored (e.g., without limitation, in the memory portion of processing unit 28), tracked over time and serve as a reference to the user for feedback. This feedback can be used for determining nutrition supplements, exercise and diet alterations.

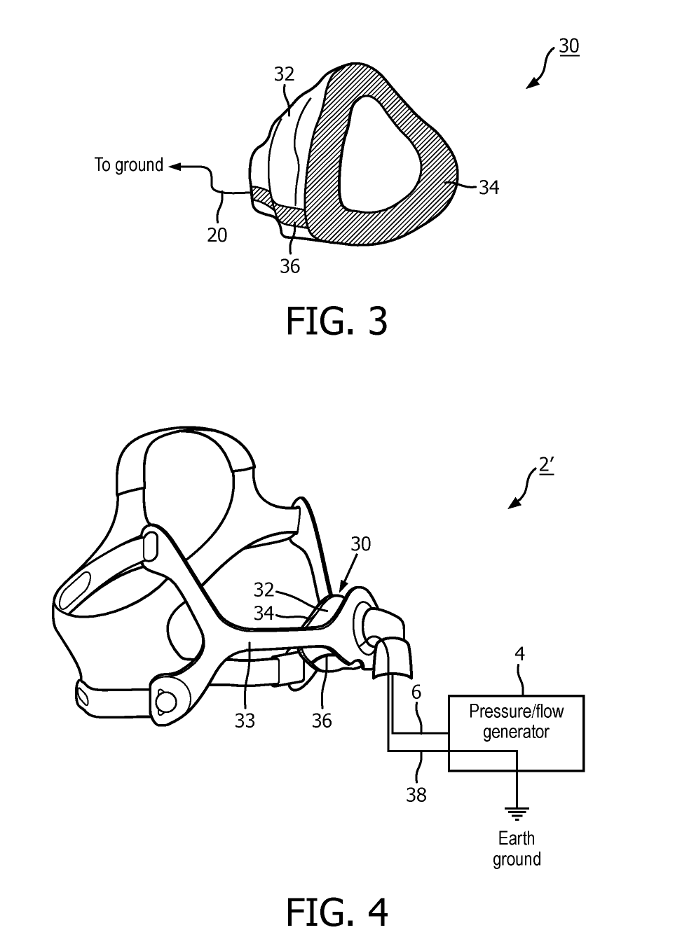

Having thus described an example of the general grounding system/arrangement of the present invention, a number of further example embodiments showing how conductive elements, such as conductive element 22 previously discussed, may be formed and/or disposed in regard to patient interface devices or components thereof will now be discussed in conjunction with FIGS. 3-7.

Referring to FIGS. 3 and 4, another example arrangement according to one particular, non-limiting exemplary embodiment in which a conductive element is disposed on the sealing portion of a patient sealing assembly is shown. More particularly, FIG. 3 illustrates a partially schematic, isometric view of the user side of a patient sealing assembly in the form of a nasal cushion 30 according to one particular, non-limiting exemplary embodiment of the present invention and FIG. 4 illustrates an airway pressure support system 2′ according to one particular, non-limiting exemplary embodiment of the present invention which includes nasal cushion 30 of FIG. 3. Nasal cushion 30 includes a body portion 32 which is structured to be coupled to a head of a user via a headgear 33. Nasal cushion 30 is adapted to receive a flow of breathing gas, such as would be provided by pressure/flow generator 4 via conduit 6. Nasal cushion 30 also includes a sealing flap 34 which is positioned and structured to sealingly engage the face of a user of cushion 30 generally about the end of the nose and the nostrils of a user. Sealing flap 34 is formed from a conductive silicone material whereas body portion 32 is formed from a traditional, non-conductive silicone. A molded lead 36 formed from conductive silicone is electrically connected to sealing flap 34 and extends therefrom for electrical connection to a suitable conductive pathway 38, e.g., similar to conductive pathway 20 of FIG. 1, which is electrically connected to Earth ground. Accordingly, sealing flap 34 functions similarly as conductive element 22 as discussed in regard to FIG. 1.

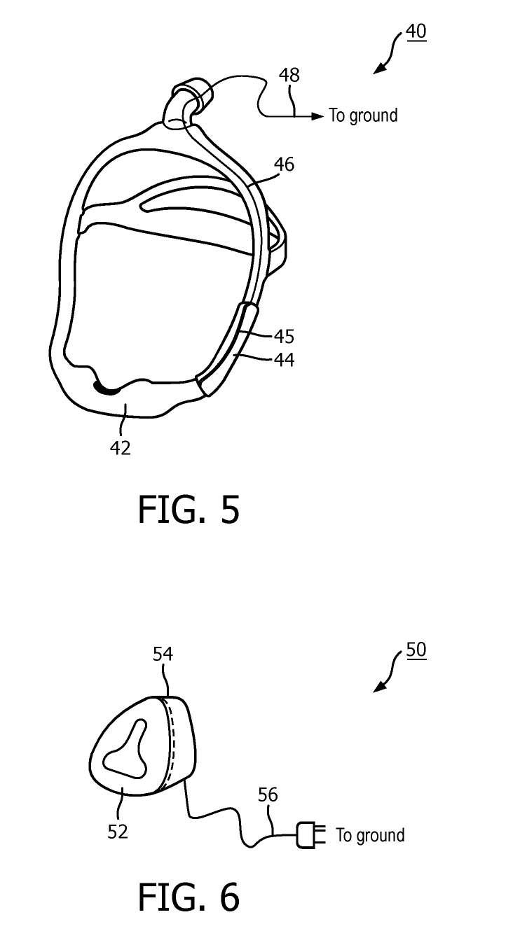

Referring to FIG. 5, an example arrangement according to one particular, non-limiting exemplary embodiment in which a conductive element is disposed on a portion of an interface device 40 that is not part of the patient sealing assembly is shown. More particularly, FIG. 5 is a partially schematic, isometric view of a patient interface device 40 which may be generally employed in a system similar to system 2, such as previously described in conjunction with FIG. 1. Similar to patient interface device 8 previously discussed in regard to FIG. 1, patient interface device 40 includes a nasal cushion 42 which is structured to sealingly engage the face of a user of patient interface device 40. Patient interface device 40 includes an electrically conductive wrap 44 (e.g., without limitation, formed from a conductive material or materials such as SHIELDEX® metalized conductive nylon fabrics—http://www.shieldextrading.net/) which is positioned to generally contact the cheek of a user of patient interface device 40. Electrically conductive wrap 44 may include one or more attachment mechanism 45, e.g., without limitation, hook and loop fastener or any other suitable mechanism which provides for conductive wrap 44 to be selectively couple to, or uncoupled from, patient interface device 40. Electrically conductive wrap 44 is connected to ground via a suitable conductive arrangement 46, e.g., without limitation, a conductive wire, which would be electrically connected to a further conductor 48 functioning in a similar manner as conductive pathway 20 of FIG. 1. Accordingly, conductive wrap 44 is a removeable element that functions similarly as conductive element 22 as discussed in regard to FIG. 1. From the foregoing, it is to be appreciated that conductive wrap 44 functions as a grounding element that may readily be retrofitted to a conventional interface device and/or which may be used in conjunction with other grounding arrangements.

FIG. 6 is a partially schematic, isometric view of a cover 50 according to one particular, exemplary embodiment of the present invention. Cover 50 is structured to fit either on only a portion of, or about generally the entirety of, a patient sealing assembly, such as, without limitation, a nasal cushion or other patient sealing assembly. In order to couple cover 50 to a patient sealing assembly, cover 50 may be provided with one or more attachment means, e.g., without limitation, an adhesive silicone layer, or other suitable mechanism. Cover 50 may include a first portion 52 formed from a conductive material (e.g., without limitation, SHIELDEX® metalized conductive nylon fabrics—http://www.shieldextrading.net/) which is positioned to directly contact the skin of a user of the patient sealing assembly and a second portion 54 which generally supports first portion 52. Alternatively, cover 50 may be formed as a single portion element formed solely from a conductive material. Cover 50 further includes a suitable conductive arrangement 56 which is structured to be electrically connected, either directly or via other means such as previously described herein, to ground. From the foregoing, it is to be appreciated that cover 50 functions as a grounding element that may readily be retrofitted to a conventional patient sealing assembly.

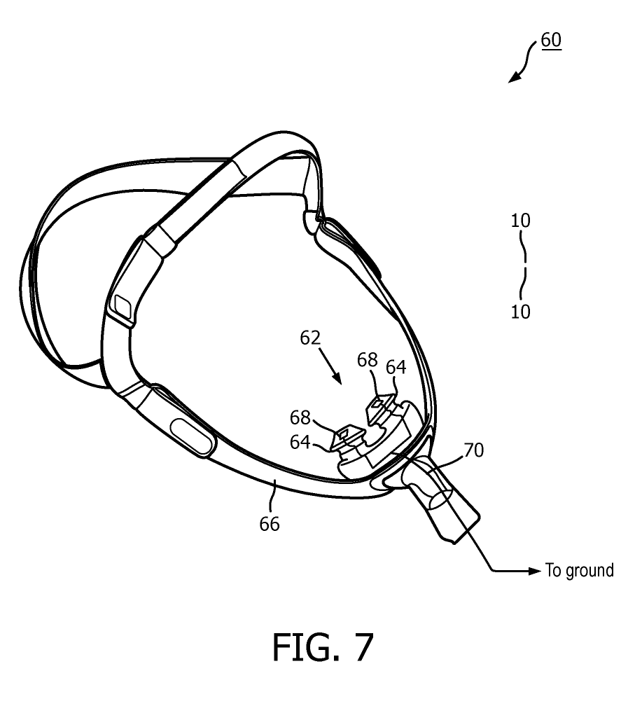

FIG. 7 is a partially schematic, isometric view of a patient interface device 60 according to one particular, exemplary embodiment of the present invention which may be generally employed in a system similar to system 2, such as previously described in conjunction with FIG. 1. Interface device 60 includes a patient sealing assembly 62, in the form of nasal pillows 64, and a headgear 66 which is structured to couple patient sealing assembly 62 to the head of a user. One or both of nasal pillows 64 may each include a conductive element 68 (shown schematically) which is structured to directly contact a user of interface device 60. Each conductive element 68 is connected to a conductive arrangement 70, which is then electrically connected to a suitable grounding arrangement, e.g., without limitation, conductive pathway 20 of FIG. 1, which is electrically connected to Earth ground.

From the foregoing examples, it is thus to be appreciated that the present invention may be applied to both new and existing arrangements for providing a flow of treatment gas to the airway of a patient and may readily be applied to generally any variation of such arrangements.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. An interface device for delivering a flow of a treatment gas to an airway of a patient, the interface device comprising:
a conductive element positioned on a surface such that the conductive element directly contacts the patient when the interface device is positioned on the patient for delivering the flow of the treatment gas to the airway of the patient; and
a conductive pathway extending from a first end which is electrically connected to the conductive element to an opposite second end which is structured to be electrically connected to earth ground.

2. The interface device of claim 1, wherein the conductive element comprises a conductive wrap selectively coupled to a portion of the interface device.

3. The interface device of claim 1, further comprising a patient sealing assembly and wherein the conductive element comprises a cover disposed on the sealing assembly.

4. The interface device of claim 3, wherein the cover is disposed only on a portion of the sealing assembly.

5. The interface device of claim 3, wherein the cover is disposed about generally the entirety of the sealing assembly.

6. A sealing assembly for use in delivering a flow of a treatment gas to an airway of a patient, the sealing assembly comprising:
a body portion structured to be coupled to a head of a user;
a sealing portion having a surface structured to sealingly engage the face of a user at or about an airway of the user; and
a conductive element positioned on the surface such that the conductive element directly contacts the patient when the interface device is positioned on the patient, wherein the conductive element is structured to be electrically coupled to ground.

7. The sealing assembly of claim 6, wherein the sealing portion is formed from a conductive material and wherein the conductive element is a portion of the sealing portion.

8. The sealing assembly of claim 7, wherein the conductive material comprises a conductive silicone.

9. The sealing assembly of claim 6, wherein the body portion is formed from a non-conductive material other than a lead formed therein which is formed from a conductive material which is electrically connected to the conductive element.

10. The sealing assembly of claim 6 wherein the conductive element comprises a cover (50) disposed on the sealing assembly.

11. The sealing assembly of claim 10, wherein the cover is disposed only on a portion of the sealing assembly.

12. The sealing assembly of claim 10, wherein the cover is disposed about generally the entirety of the sealing assembly.

13. An airway pressure support system for use in delivering a flow of breathing gas to the airway of a patient, the pressure support system comprising:
a pressure/flow generating device which is structured to generate the flow of breathing gas;
a patient interface device structured to be disposed on the head of the patient, the patient interface device comprising:
a headgear for securing the patient interface device to the head of the patient; and
a patient sealing assembly which is structured to sealingly engage the face of the patient at or about an airway of the patient;
a delivery conduit coupled between the pressure/flow generating device and the patient interface device, the delivery conduit structured to communicate the flow of breathing gas from pressure generating device to the patient interface device;
a conductive element positioned on a surface of the patient interface device such that the conductive element directly contacts the patient when the interface device is positioned on the patient; and
a conductive pathway which extends from the conductive element to a second end which is structured to be electrically connected to earth ground.

14. The airway pressure support system of claim 13, wherein the conductive pathway is a portion of a grounding system which also comprises a processing unit;
wherein the conductive pathway includes a first resistor (R1), a switch (S1), and a second resistor (R2) which are all electrically connected in series; and
wherein the switch is selectively switched between an open position and a closed position by the processing unit.

15. The airway pressure support system of claim 14, wherein the processing unit is associated with the control/operation of the pressure/flow generating device.
